(19) United States
(12) Patent Application Publication
(10) Pub. No.: US 2022/0110641 A1
(43) Pub. Date: Apr. 14, 2022

(54) METATARSOPHALANGEAL (MTP) FUSION PLATE DEVICE WITH GRAFT HOLDING AND GRAFT HARVESTING INSTRUMENT

(71) Applicants: Thomas Zink, San Antonio, TX (US); Kyle Vaughn, Phoenix, AZ (US); Jason Kayce, Phoenix, AZ (US)

(72) Inventors: Thomas Zink, San Antonio, TX (US); Kyle Vaughn, Phoenix, AZ (US); Jason Kayce, Phoenix, AZ (US)

(21) Appl. No.: 17/497,109

(22) Filed: Oct. 8, 2021

Related U.S. Application Data
(60) Provisional application No. 63/089,575, filed on Oct. 9, 2020.

(51) Int. Cl. A61B 17/16; A61B 17/15; A61B 17/17; A61B 17/80; B33Y 80/00

(52) U.S. Cl. CPC A61B 17/1635; A61B 17/151; A61B 17/1775; A61B 2017/00526; A61B 17/8061; B33Y 80/00; A61B 17/1697

(57) ABSTRACT

A graft-harvesting instrument and MTP fusion device are disclosed to allow for easy and safe removal of a section of the calcaneus to be used as autograft in the fusion of the MTP joint. The graft-harvesting instrument allows a surgeon to place a barrier between the saw and the vital anatomy on the medial side of the calcaneus. The MTP fusion device allows for the exact placement of the autograft into the MTP joint. The graft-harvesting instrument allows for a very consistent piece of autograft to be harvested. The graft-retaining sleeve component of the MTP fusion device positions that autograft in place for optimum fusion of the MTP joint.

### CROSS-REFERENCE TO RELATED APPLICATION

The present application claims priority to, and the benefit of, U.S. Provisional Application No. 63/089,575, which was filed on Oct. 9, 2020, and is incorporated herein by reference in its entirety.

### FIELD OF THE INVENTION

The present invention relates generally to a medical device and instrument for use in treatment of the foot, particularly those relating to arthritic conditions. More particularly, the present disclosure relates to a graft harvesting instrument which allows for the easy extraction of autograft bone from the calcaneus area and a first metatarsophalangeal (“MTP”) fusion device and that is designed to hold the autograft bone in place to promote lengthening of the hallux (great or “big” toe) and fusion of the joint. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

A first metatarsophalangeal joint fusion is typically performed in the setting of painful arthritis of the first MTP joint—also known as late stage or severe hallux rigidus. An MTP joint fusion can also be utilized as a deformity corrective procedure in patients with severe bunions, also known as hallux valgus.

A bunion (hallux valgus) is a multi-planar angular deformity of the first MTP joint. The deformity often causes abnormal fitting of shoes, discomfort in walking or other activities and irritation of the skin in places where the toe contacts the shoe or other footwear, which often leads to inflammation and pain. A bunion is caused by a malalignment of the first metatarsal and the proximal phalanx of the hallux. This alters the distribution of ground reactive forces throughout the human gait cycle which can lead to irritation, calluses, blistering and ulceration of the skin, as well as difficulties in walking.

Malalignment of the first MTP joint for a sustained period of time will also eventually lead to degenerative changes or arthritis of the joint, thereby leading to pain, stiffness, inflammation and difficulty maintaining a normal gait pattern.

The goal of fusion surgery is to align the hallux in a functional position and fuse the MTP joint. This eliminates painful motion created by the arthritic joint surfaces. To achieve fusion of the joint, any residual cartilage is removed from both joint surfaces and the underlying subchondral bone is prepared for fusion with fenestration. Alternatively, drilling the bone to create tiny channels for bleeding to occur provides lubrication to the area. The hallux is positioned in a manner which optimizes gait function and the ability to wear a shoe without difficulty. This is typically done with the hallux positioned so it gently presses the weight-bearing surface when standing. The prepared joint is typically fused with one or two compression screws placed obliquely across the joint, with a neutralization plate spanning the fusion site dorsally.

Typically, in order to correct a bunion deformity, an osteotomy is made in the metatarsal, which allows the head of the metatarsal to be translated laterally in the transverse plane which causes realignment of the MTP joint. The osteotomy is fixated with cortical compression screws or pins.

Specifically, an incision is made at the dorsal joint surface. The residual cartilage is extracted or removed from the metatarsal head and phalangeal base. The surgeon will typically create a “ball and socket” configuration of the surfaces, using a variety of instrumentation and procedures. With the joint surfaces prepared and positioned appropriately, 1 or 2 compression screws or other fasteners are placed across the joint and the dorsal plate is applied and secured in position. After implantation of the metallic hardware, the surgical site is closed with retained sutures. The foot and lower leg are then typically placed in a splint to hold the foot and lower leg in position while healing occurs, and to allow time for the plate and fasteners to set in position.

While traditional methods of primary first MTP joint fusion have proven to be effective, there are identifiable shortcomings when this type of fixation is utilized while attempting to maintain proper alignment of the hallux and restore anatomic length of the first ray, after a failed prior attempt at fusion or the use of a joint-sparing implant. A shortening of the toe or area is created as it is necessary to resect non-viable bone and tissue at the non-union site in order to create an environment which is conducive to healing, and bony bridging or on growth.

Typically, a block of bone, either transplanted from the surgical patient (autograft), or cadaveric bone (allograft) is utilized. Autograft tissue contains osteogenic tissue that has been shown to aid the development of hard osseous tissue or bone. Currently, harvesting the calcaneus material is a very time-consuming procedure. The surgeon uses a saw or other cutting implement to cut the near cortex of the bone, but must be careful to not cut the far cortex when attempting to remove sufficient material for use. After the initial cut with a saw, the surgeon must use osteotomes to cut the far cortex and wedge the autograft section from the calcaneus slowly and carefully into position. The autograft section can be damaged in the process of wedging it from the calcaneus, which may result in additional material needing to be harvested or obtained.

Once the autograft section is removed, it is placed into the MTP joint and then a dorsal plate and/or compression screws or fasteners are used for fixation on the dorsal surface of the bone. However, the graft can lose shape and be very hard to hold in place to optimize fusion of the joint. An interfrag screw is sometimes placed through the graft, but this can fracture or break apart the graft, and cause loss of structural integrity of the construct. Further, these methods generally do not create an ideal lattice structure to facilitate bony ingrowth.

Additionally, the traditional use of the neutralization plate creates increased bulk in the soft tissue layer, which can be a source of irritation with shoes or other footwear. Correct positioning of the hallux in the sagittal plane is critical. The use of a straight plate to assist with the fusion can be difficult. Frequently, the plate needs to be bent, shaped or configured to accommodate the appropriate position of the hallux. Bending or shaping the plate can lead to premature failure, as well as affect the way the affixing locking screws lock into the plate, and potentially impacting the structural integrity of the plate.

Thus, there exists a need for an improved graft harvesting instrument and MTP device for fusing an MTP joint (primary or revision) or correcting a severe bunion deformity that achieves restoration of length during a revision surgery and provides an appropriate structural lattice for bony ingrowth. The present invention discloses a graft-harvesting instrument to allow for easy extraction of autograft bone from the calcaneus, and an MTP fusion device which stabilizes the fusion site, holds autograft bone in place to promote lengthening of the toe and fusion of the joint. The present invention provides for restoration of lost length of the first ray, secondary to failed fusion attempts or explantation of a joint implant from an attempted joint salvage procedure. Further, the graft harvesting instrument includes a pair of handles that are removably-secured to a pair of L-shaped components. The pair of L-shaped components are positioned over the calcaneus to act as a barrier between the saw and the vital anatomy on the medial side of the calcaneus. The MTP fusion device includes an MTP plate with an osteo-integrative mesh retaining sleeve component, sized and configured to retain the autograft bone section harvested from the calcaneus via the graft harvesting instrument. Thus, the graft harvesting instrument and MTP fusion device provide for a more secure fusion environment, increased capacity for bone ingrowth and lengthening of the toe as compared to traditional methods.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some general concepts in a simplified form as a prelude to the more detailed description that is presented later.

The subject matter disclosed and claimed herein, in one aspect thereof, comprises a graft-harvesting instrument and MTP device for fusing an MTP joint to restore length during a revision surgery, and provide an appropriate structural lattice for bony ingrowth. Specifically, the graft-harvesting instrument allows for the easy extraction of autograft bone from the calcaneus. The graft-harvesting instrument includes a pair of handles which are removably-secured to a pair of L-shaped components (or cut blade components). The pair of handles pivot together and apart to push the L-shaped components together or move them apart. The L-shaped components are spread apart and then positioned over the calcaneus to act as a barrier between the saw and the vital anatomy on the medial side of the calcaneus.

In a preferred embodiment, the graft harvesting instrument further includes a saw cut guide that is slid onto the L-shaped component via a T-slot or other connector and fixated with pins, screws, fasteners or K-wires. The pair of handles of the graft-harvesting instrument can then be pivoted out of the way to allow for easy saw or cutter insertion. Once the surgeon is done cutting, the pair of handles is then pivoted back down and squeezed together so the surgeon can easily pull the autograft out of the calcaneus in one piece and one fluid motion.

The MTP fusion device is designed to hold the autograft bone in place to promote lengthening of the toe and fusion of the joint. The MTP fusion device includes an MTP plate with a retaining sleeve component that is sized and configured to retain the autograft bone section which was harvested from the calcaneus via the graft harvesting instrument. The MTP plate has an elongated plate section extending along a length and having a first end and a second end. The first end has a terminal area with a compression slot and opposing first and second ears with threaded through holes, and the second end has a terminal area with a screw hole and opposing first and second ears with threaded through holes. The plate further includes a transverse central axis about which the plate displays a substantial mirror symmetry.

In another embodiment, the retaining sleeve component of the MTP fusion device is comprised of an osteo-integrative mesh. The osteo-integrative mesh provides for an increased capacity for bone ingrowth and a more secure fusion between the connected elements. Typically, the graft protrudes from either side of the retaining sleeve component to assure contact with bone on different surfaces of the graft.

In another embodiment, the graft-harvesting instrument is machined of medical-grade titanium and the MTP fusion device is manufactured as an additively-printed titanium component which can be sized and configured depending on the measurements or constraints of the particular procedure.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a side perspective view of one potential embodiment of the graft-harvesting instrument of the present invention disclosing placement over the calcaneus in accordance with the disclosed architecture;

FIG. 2 illustrates a front perspective view of one potential embodiment of the graft-harvesting instrument of the present invention with the saw cut guide component attached in accordance with the disclosed architecture;

FIG. 3 illustrates a side perspective view of one potential embodiment of the graft-harvesting instrument of the present invention with the pair of handles pivoted out of the way for saw insertion in accordance with the disclosed architecture;

FIG. 4 illustrates a bottom perspective view of one potential embodiment of the MTP plate of the present invention and having a retaining sleeve component in accordance with the disclosed architecture;

FIG. 5 illustrates a side perspective view of one potential embodiment of the MTP plate of the present invention and having a retaining sleeve component with the mesh removed in accordance with the disclosed architecture;

FIG. 6 illustrates a side perspective view of one potential embodiment of the MTP plate of the present invention with the graft in place within the retaining sleeve component in accordance with the disclosed architecture;

FIG. 7 illustrates a top perspective view of one potential embodiment of the MTP plate in use and secured across an MTP joint in accordance with the disclosed architecture; and

FIG. 8 illustrates a block diagram of one potential method of manufacturing the MTP plate of the present invention in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention, and do not limit the scope of the invention. Additionally, an illustrated embodiment need not have all the aspects or advantages shown. Thus, in other embodiments, any of the features described herein from different embodiments may be combined.

The present invention discloses a graft-harvesting instrument and MTP fusion device to create a more secure fusion environment, increased capacity for bone ingrowth, and lengthening of the toe as compared to traditional or conventional methods. The graft-harvesting instrument allows for easy and safe removal of a section of the calcaneus to be used as autograft. The instrument allows a surgeon to place a barrier between the saw or cutting implement and the vital anatomy on the medial side of the calcaneus. The MTP fusion device allows for the exact placement of the autograft into the MTP joint. The graft-harvesting instrument allows for a very consistent piece of autograft to be harvested. The graft-retaining sleeve component of the MTP fusion device positions that autograft in place for optimum fusion of the MTP joint.

Further, the graft-harvesting instrument has a pair of handles and a pair of L-shaped components that are positioned over the calcaneus to act as a barrier between the saw or cutting tool and the vital anatomy on the medial side of the calcaneus. The MTP fusion device includes an MTP plate with an osteo-integrative mesh retaining sleeve component, sized to retain the autograft bone section harvested from the calcaneus via the graft-harvesting instrument.

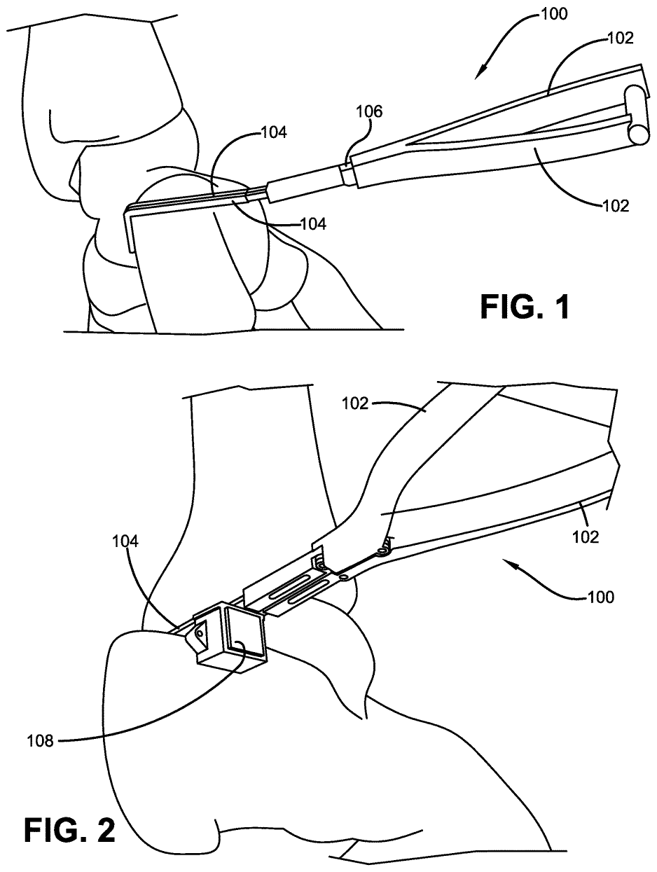

Referring initially to the drawings, FIGS. 1-3 illustrate the graft-harvesting instrument 100 which allows for the easy and safe removal of a section of the calcaneus to be used as autograft. The graft-harvesting instrument 100 can be any suitable size, shape and configuration as is known in the art without affecting the overall concept of the invention. One of ordinary skill in the art will appreciate that the shape and size of the graft-harvesting instrument 100 as shown in FIGS. 1-3 is for illustrative purposes only, and many other shapes and sizes of the graft-harvesting instrument 100 are well within the scope of the present disclosure. Although dimensions of the graft-harvesting instrument 100 (i.e., length, width, and height) are important design parameters for good performance, the graft harvesting instrument 100 may be any shape or size that ensures optimal performance during use, and may even be customized to fit the exact specifications/measurements of the patient's foot.

Additionally, the graft-harvesting instrument 100 has a pair of handles 102 operable to be squeezed together and spread apart by a user. The graft harvesting instrument 100 also has a pair of L-shaped components 104 which are removably-secured to the pair of handles 102, and extend outwardly from the end of the handles. Typically, the pair of L-shaped components 104 are removably-secured to the pair of handles 102 via a push button connector 106 or any other securing means as is suitable. The pair of handles 102 act to pivot or move the pair of L-shaped components 104 into an open and closed position. Specifically, spreading apart the pair of handles 102 acts to open the pair of L-shaped components 104 or spread them apart so that a surgeon may position the pair of L-shaped components 104 over the calcaneus to create a barrier that protects the nerves and vessels on the medial side of the calcaneus. The spread-apart pair of L-shaped components 104 forms a barrier which allows the surgeon to use a saw or other tool to cut through the calcaneus without fear of damaging vital anatomy on the medial side.

Further, the graft-harvesting instrument 100 has a saw cut guide 108 that is slid onto the pair of L-shaped components 104 when the components 104 are in an open position. Specifically, the saw cut guide 108 is secured to the pair of L-shaped components 104 via a T-slot (not shown) or other suitable connector. Once the saw cut guide 108 is secured to the pair of L-shaped components 104, it is fixated with pins or K-wires or any other suitable fixating means as is known in the art. The saw guide 108 can be positioned in any suitable location along the L-shaped components depending on the location that the cutting is to take place.

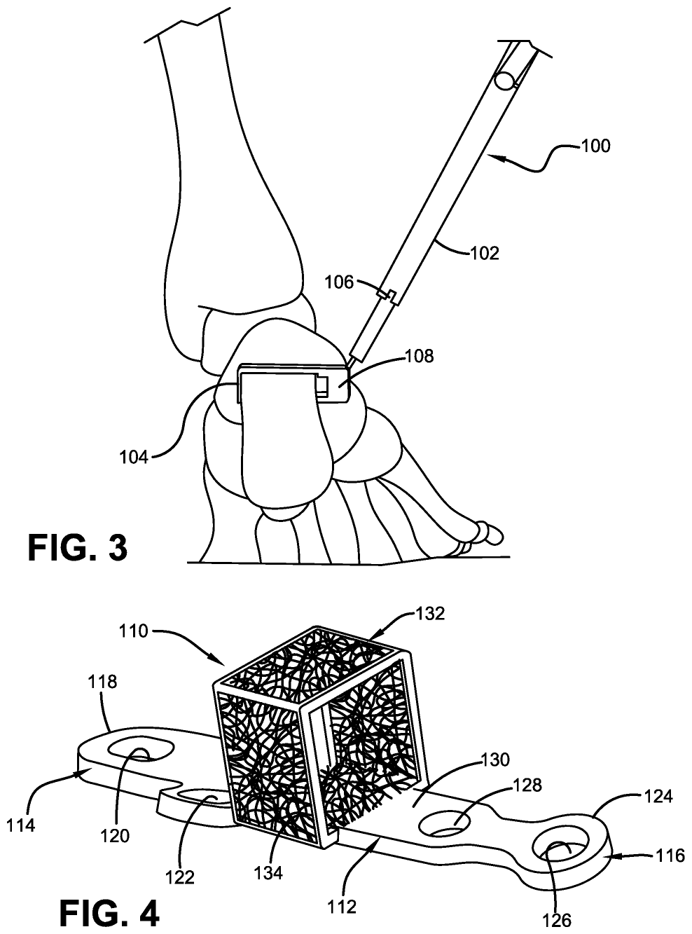

As shown in FIG. 3, the pair of handles 102 of the graft-harvesting instrument 100 can then be pivoted out of the way to allow for easy saw insertion. This allows the surgeon access to the saw cut guide 108. Once the surgeon is done cutting, the pair of handles 102 are then pivoted back down and squeezed together, which pushes the pair of L-shaped components 104 together or in a closed position. Once the pair of L-shaped components 104 is in a closed position, the surgeon can easily pull or remove the autograft out of the calcaneus in one piece with one fluid motion. Thus, the pair of L-shaped components 104 needs to be in an open or first position for cutting, and then in a second or closed position to extract the autograft once the cutting is complete.

In a preferred embodiment, the graft-harvesting instrument 100 is machined of medical-grade titanium. Further, the graft-harvesting instrument 100 is manufactured with medical-grade titanium but can be manufactured with any other suitable metal as is known in the art, as long as the metal is medical grade.

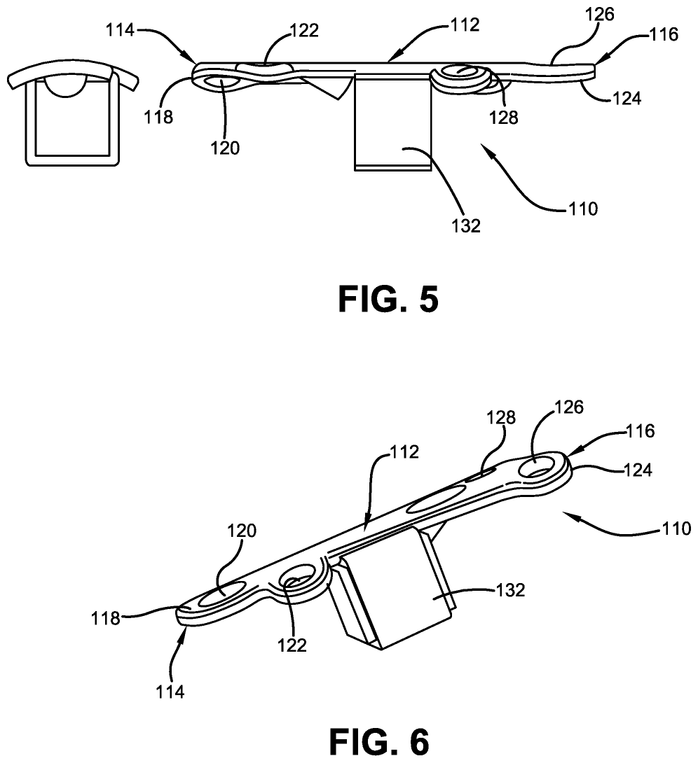

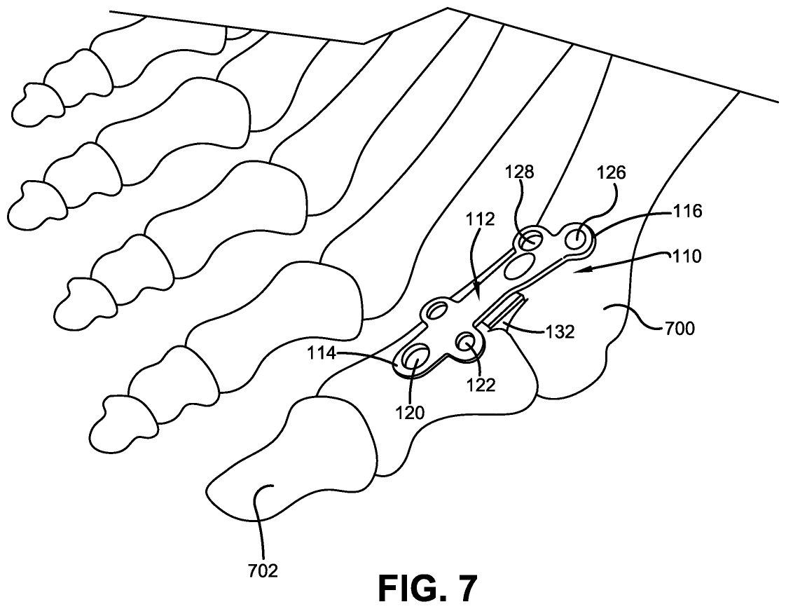

Once the graft harvesting instrument 100 harvests a piece of autograft, the MTP fusion device 110 positions that autograft in place for optimum fusion of the MTP joint. Referring initially to the drawings, FIGS. 4-7 illustrate the MTP fusion device 110 which is designed to hold the autograft bone in place to promote lengthening of the toe and fusion of the joint. The MTP fusion device 110 can be any suitable size, shape and configuration as is known in the art without affecting the overall concept of the invention. One of ordinary skill in the art will appreciate that the shape and size of the MTP fusion device 110 as shown in FIGS. 4-7 is for illustrative purposes only, and many other shapes and sizes of the MTP fusion device 110 are well within the scope of the present disclosure. Although dimensions of the MTP fusion device 110 (i.e., length, width and height) are important design parameters for good performance, the MTP fusion device 110 may be any shape or size that ensures optimal performance during use, and may even be customized to fit the exact specifications or measurements of the patient's first metatarsal.

The MTP fusion device 110 is an intramedullary fixation device which includes a plate 112 which is capable of fixation of bone at the first metatarsophalangeal joint. Further, the plate 112 extends along a length, and has a first end 114 and a second end 116. The first end 114 has a terminal area 118 with a compression slot 120 and opposing first and second ears 122, the first and second ears 122 each have a threaded through hole for receiving a fastener. Further, the second end 116 also includes a terminal area 124 with a screw hole 126 and opposing first and second ears 128, the first and second ears 128 each having a threaded through hole. Wherein, the opposing ears 122 on the first end 114 and the opposing ears 128 on the second end 116 are offset to each other relative to the length of the plate 112. The first and second ears 122 and 128 and the screw hole 126 all have internal threads to accept screws or other fasteners as are known in the art to create locking holes to secure the plate 112 to the bone. Further, the compression slot 120 includes an edge that has an incline that slopes downward toward the transverse medial axis of the plate 112, when a screw is secured within the compression slot 120, the osteotomy is compressed together.

Additionally, the plate 112 has an inner surface 130 which includes a first axis and which forms an angle in the dorsal direction of between 0°-10°, and which forms an angle in the valgus direction of between 0°-20° degrees. Further, the plate 112 has a second axis as well as a first axis, and the first and second axis being at an angle from about 0° to about 10° from each other in the dorsal direction and in the valgus direction from about 0° to about 20° from each other. Further, the plate 112 has a transverse central axis about which the plate 112 displays at least substantial mirror symmetry.

Moreover, the plate 112 of the MTP fusion device 110 includes a retaining sleeve component 132 designed to hold the autograft bone 600 in place to promote lengthening of the toe and fusion of the joint. Specifically, the retaining sleeve component 132 is sized to retain the autograft bone section 600 harvested from the calcaneus via the graft harvesting instrument 100. Typically, the autograft 600 protrudes from either side of the retaining sleeve component 132 to assure contact with bone. Thus, any compression on the joint will be directly placed on the graft 600. Having the graft 600 under compressive load is intended to promote the bone to grow faster and stronger as well as to hold the graft in position.

Further, the retaining sleeve component 132 of the MTP fusion device 110 has an osteo-integrative mesh 134. The osteo-integrative mesh 134 which is positioned generally centrally of the plate, provides for an increased capacity for bone ingrowth and a more secure fusion. The osteo-integrative mesh 134 can be shaped as a geometric design comprising shapes such as crosses, squares, and/or triangles in every direction, or shaped more of an organic design where the indices are random and protrude in all directions, at different angles and thicknesses. This osteo-integrative mesh 134 promotes bone growth within and around the surgical site, creating a more secure fusion of the MTP joint.

In a preferred embodiment, the MTP fusion device 110 is manufactured using additive manufacturing (AM) techniques and grown as one part. Specifically, the MTP fusion device 110 is additively printed, and able to be manufactured in a variety of sizes as well as to be customizable to fit the exact specifications or measurements of the patient's foot. Further, the MTP fusion device 110 is additively printed with titanium, but can be additively printed with any other suitable metal as is known in the art, as long as the metal is medical grade and able to be additively printed.

In operation, as shown in FIG. 7, a surgeon spreads apart the pair of handles 102 of the graft-harvesting instrument 100 to open or spread apart the pair of L-shaped components 104 to create a barrier that protects the nerves and vessels on the medial side of the calcaneus. Specifically, the surgeon positions the pair of L-shaped components 104 over the calcaneus to create a barrier, which allows the surgeon to use a saw or other tool to cut through the calcaneus without fear of damaging vital anatomy on the medial side.

Once the graft-harvesting instrument 100 is in place, a saw cut guide 108 is then slid onto the pair of L-shaped components 104 when the components 104 are in an open position via a T-slot (not shown). The saw cut guide 108 is then fixated with pins or K-wires. The pair of handles 102 of the graft harvesting instrument 100 is then pivoted out of the way to allow for easy saw insertion. This allows the surgeon access to the saw cut guide 108. Once the surgeon is done cutting, the pair of handles 102 is then pivoted back down and squeezed together, which pushes the pair of L-shaped components 104 together or in a closed position. Once the pair of L-shaped components 104 is in a closed position, the surgeon can easily pull the autograft out of the calcaneus in one piece with one fluid motion.

Once the graft-harvesting instrument 100 harvests a piece of autograft, the surgeon positions the piece of autograft within the retaining sleeve component 132 of the MTP fusion device 110 for optimum fusion of the MTP joint. The MTP fusion device 110 is then applied to the first metatarsal 700 of the foot. The goal of surgery is to make the great toe joint solidly aligned and immobile. This eliminates much of the pain associated with the arthritic joint, since there will now be no motion through the arthritic joint. Specifically, an incision is made on top of the big toe. Any remaining cartilage is cleared away to allow the two bones to heal or fuse together. To fuse the great toe joint, any remnant cartilage on the arthritic joint surface is also removed, and the surgeon uses a combination of instruments and tools to shape each bone for a perfect fit, which prepares the underlying bone for fusion.

Then, the plate 112 of the MTP fusion device 110 is positioned over the first metatarsal 700. The joint is then positioned in a manner that maximizes walking ability and maintains acceptable clinical alignment. This is traditionally done with the toe positioned so that it just gently touches the ground in a weight-bearing position. Once the joint is correctly positioned, a screw or other fastener (not shown) is driven through the screw hole 126 on the second end 116 into the phalanx 702, and then a screw or other fastener (not shown) is driven through the compression slot 120 on the first end 114 into the metatarsal 700 to compress the osteotomy together. Once the desired compression is achieved, screws are driven through the opposing first and second ears 122 and 128 to further secure the MTP fusion device 110 in position between the first metatarsal 700 and the distal phalanx 702. Typically, the autograft protrudes from either side of the retaining sleeve component 132 to assure contact with bone. Thus, any compression on the joint will be directly placed on the graft. Having the graft under compressive load will promote the bone to grow faster and stronger.

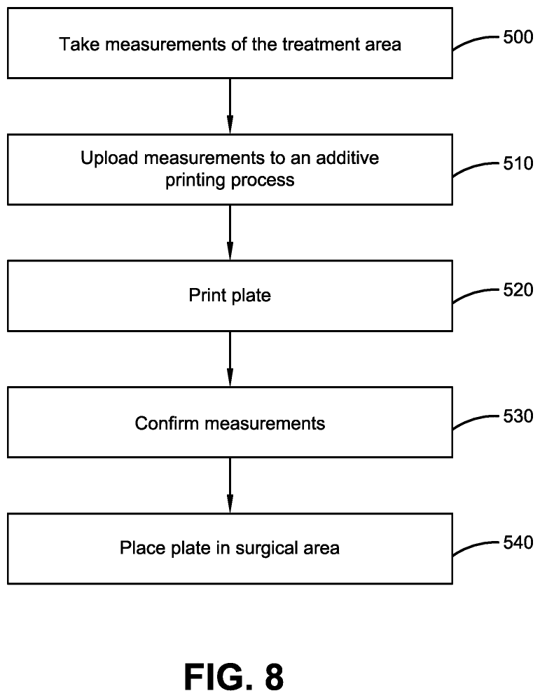

FIG. 8 illustrates a block diagram of one potential method of manufacturing the MTP plate of the present invention in accordance with the disclosed architecture. The method begins at step 500 with obtaining measurements for the fusion device or plate. The measurements are uploaded at step 510 to a surgical implant printing device such as, but not limited to, a 3-D metal printing device. Then, at step 520, the plate or fusion device is printed, and the measurements of the plate are confirmed, with the initial measurements entered into the system at step 530. Once the set of measurements is confirmed, then at step 540 the plate is inserted into position during a surgical procedure.

Certain terms are used throughout the following description and claims to refer to particular features or components. As one skilled in the art will appreciate, different persons may refer to the same feature or component by different names. This document does not intend to distinguish between components or features that differ in name but not structure or function. Also, notwithstanding the forgoing, the present invention and its various components can be of any suitable size and configuration as is known in the art without affecting the overall concept of the invention, provided that they accomplish the above-stated objectives. One of ordinary skill in the art will appreciate that the size, configuration and material of the invention and its various components as shown in the FIGS. are for illustrative purposes only, and that many other sizes and shapes of the invention and its various components are well within the scope of the present disclosure. Although the dimensions of the invention and its components are important design parameters for user convenience, the invention and its components may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A graft harvesting tool comprising:
a pair of handles moveable from a first position to a second position;
a first component and a second component positioned at each end of the pair of handles; and
a saw guide disposed over a portion of the first and second components, wherein the saw guide provides for accurate cutting of a bone when the pair of handles is provided in the first position.

2. The graft harvesting tool as recited in claim 1, wherein the first position is an open position and the second position is a closed position.

3. The graft harvesting tool as recited in claim 1, wherein the saw guide is positionable on the first and second components to define a cutting area.

4. The graft harvesting tool as recited in claim 3, wherein the saw guide is held in position by at least one of a pin or a wire.

5. The graft harvesting tool as recited in claim 1, wherein the first and second components are L-shaped.

6. The graft harvesting tool as recited in claim 4, wherein the wire is a K-wire.

7. The graft harvesting tool as recited in claim 3, wherein a graft is removed from the cutting area when the pair of handles are in the closed position.

8. The graft harvesting tool as recited in claim 1, wherein the pair of handles can be pivoted upwards away from the saw guide during cutting.

9. A metatarsophalangeal (MTP) fusion device comprising:
a plate for use in a MTP fusion procedure, wherein the plate is comprised of a first end, a second end, an inner surface, an outer surface and a compression slot; and
a retaining sleeve component comprised of an osteo-integrative mesh.

10. The metatarsophalangeal (MTP) fusion device as recited in claim 9, wherein the osteo-integrative mesh is comprised of a geometric design pattern or a random design pattern.

11. The metatarsophalangeal (MTP) fusion device as recited in claim 9, wherein the compression slot is positioned over a graft to compress the graft into position.

12. The metatarsophalangeal (MTP) fusion device as recited in claim 9, wherein the plate has an angle on the inner surface in a dorsal direction of between 0° and 10°.

13. The metatarsophalangeal (MTP) fusion device as recited in claim 9, wherein the plate has an angle on the inner surface in a valgus direction of between 0° and 20° degrees.

14. The metatarsophalangeal (MTP) fusion device as recited in claim 9, wherein each of the first and second ends of the plate comprises a pair of ears extending generally outwardly from a pair of sides of the plate.

15. The metatarsophalangeal (MTP) fusion device as recited in claim 9, wherein the plate comprises a plurality of through holes to receive one or more fasteners.

16. The metatarsophalangeal (MTP) fusion device as recited in claim 9, wherein the osteo-integrative mesh is positioned in a central portion of the plate.

17. The metatarsophalangeal (MTP) fusion device as recited in claim 9, wherein the compression slot comprises an edge having an incline that slopes downward toward a transverse medial axis of the plate.

18. The metatarsophalangeal (MTP) fusion device as recited in claim 9, wherein the retaining sleeve component holds the plate in a desired position.

19. A method of making a metatarsophalangeal (MTP) fusion device comprising the steps of:
collecting a plurality of measurements for use in create a MTP fusion device;
loading the plurality of measurements into a system for additively printing the MTP fusion device;
printing the MTP fusion device;
confirming the plurality of measurements of the MTP fusion device; and
installing the MTP fusion device during a surgical procedure.

20. The method of making a metatarsophalangeal (MTP) fusion device as recited in claim 19, wherein the MTP fusion device comprises a first end, a second end, a pair of ears disposed at each of the first and second ends, a retaining sleeve component and an osteo-integrative mesh comprised of a geometric design pattern or a random design pattern.
